(19) United States
(12) Patent Application Publication
(10) Pub. No.: US 2006/0099580 A1
(43) Pub. Date: May 11, 2006

(54) Methyl-cpg binding domain protein 2 homologs

(76) Inventors: Corena T McManus, Salt Lake City, UT (US); David A Jones, Salt Lake City, UT (US)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/510,162

(22) PCT Filed: Apr. 7, 2003
(86) PCT No.: PCT/US03/10631

§ 371 Date: Dec. 20, 2005

Related U.S. Application Data
(60) Provisional application No. 60/369,851, filed on Apr. 5, 2002.

(51) Int. Cl. C12Q 1/68; C07H 21/04; C12P 21/06; C12N 9/22

(52) U.S. Cl. 435/6; 536/23.2; 435/69.1; 435/199; 435/320.1; 435/325

(57) ABSTRACT

Two novel proteins, MBD2-CTH1 and MBD2-CTH2, are described that share extensive sequence homology to MBD2. These novel proteins lack MBDs and do not bind methylated DNA, but do, however, localize to the nucleus and assemble into transcriptional repressor complexes. These properties of MBD2-CTH1 and MBD2-CTH2 demonstrate that one or both of the homologs acts as dominant negative inhibitors of MBD2. MBD2-CTH1 and MBD2-CTH2, thus, provide new reagents for inhibiting methylation-dependent repression and thereby treat various pathologies, e.g. colon cancer.

This application claims priority to U.S. Provisional Application Ser. No. 60/369,851 filed Apr. 5, 2002 which is hereby incorporated by reference in its entirety.

### BACKGROUND

Colon cancer is the third most common cancer diagnosed in the United States. This year it is estimated that 98,200 people will be diagnosed with colon cancer and 48,100 deaths will occur due to the disease (American Cancer Society 2001). Understanding how colon cancer forms is a fundamental step toward developing drugs to treat the disease. In studying the progression of colon cancer, many genetic alterations are needed to change normal colon cells into invasive cancer. Genes involved in preventing tumor formation or growth, known as tumor suppressor genes, are often mutated thereby eliminating their function. Although mutation or deletion are the most common genetic events that can impair tumor suppressor gene function, methylation of the promoter within the gene is an alternative mechanism for inactivation.

The importance of methylation in colon cancer is becoming increasingly apparent. Tumor suppressor genes important for both sporadic and familial colon cancer have been found to be highly methylated in colon cancers compared to benign tumors (Cunningham et al. 1998; Herman et al. 1995; Herman et al. 1998; Hiltunen et al. 1997; Kane et al. 1997; Veigl et al. 1998). Unlike mutations and deletions, however, silencing a gene by methylation is a reversible process that can be targeted with drugs, like 5-aza-2′-deoxycytidine (aza-dC), to facilitate reactivation of gene function (Herman et al. 1994; Herman et al. 1998; Merlo et al. 1995). This suggests that demethylating agents could restore the expression of methylation-silenced genes, perhaps reversing the tumorigenic state. As such, proteins involved in methylation silencing have become attractive targets for the development of anti-cancer drugs.

Current models indicate that methylation-dependent gene silencing relies on a family of proteins containing methyl-CpG binding domains (MBDs). The family consists of five proteins, MeCP2 (methyl-CpG binding protein 2), MBD1, MBD2, MBD3, and MBD4 (Cross et al. 1997; Hendrich and Bird 1998; Lewis et al. 1992). These proteins repress gene activation by recruiting protein complexes containing histone deacetylases (HDACs). This results in compacting DNA in a way that impairs gene activation. Several repressor complexes contain methyl-CpG binding proteins as core subunits or auxiliary subunits. The nucleosome remodeling and histone deacetylation (NuRD) complex contains MBD3 as a core component and can associate with MBD2 (Wade et al. 1999; Zhang et al. 1999). This interaction is most likely mediated through MBD3, since MBD2 and MBD3 can interact with each other (Tatematsu et al. 2000). In addition, the Sin3 repressor complex associates with both MeCP2 (Jones et al. 1998) and MBD2 (Boeke et al. 2000) and MeCP1, a repressor complex known to bind methylated DNA, also contains MBD2 (Ng et al. 1999). The association between transcriptional repressor complexes and methyl-CpG binding proteins could result in the recruitment of the repression complexes to methylated DNA with the ultimate outcome of decreased transcription of the target gene.

### SUMMARY OF THE INVENTION

Accordingly, it is an object of the invention to provide molecules that do not bind methylated DNA, but are able to interfere with the ability of methyl-CpG binding domains to bind methylated DNA.

It is a further object of the invention to provide medicaments and methods for treating pathologies characterized by down-regulation of tumor repressor genes.

It is still another object of the invention to provide medicaments and methods for reactivating methylation-silenced genes in a subject suffering from a disease characterized by down-regulation of tumor repressor genes.

These and other methods, as more fully described herein, are provided for by the present invention.

In a compositional sense, the invention provides purified nucleic acid molecules that contain at least 200 contiguous nucleotides of a nucleic acid sequence encoding MBD2-CTH1 (SEQ ID NO: 2) or MDB2-CTH2 (SEQ ID NO: 4), or a complementary strand of one of the foregoing nucleic acid sequences. The invention also provides purified polypeptides that contain at least 67 contiguous amino acids of the amino acid sequence of MBD2-CTH1 (SEQ ID NO: 2) or MDB2-CTH2 (SEQ ID NO: 4). More specifically, the invention provides a purified nucleic acid molecule containing a nucleic acid sequence which encodes the amino acid sequence of MBD2-CTH1 (SEQ ID NO: 2)or MDB2-CTH2 (SEQ ID NO: 4).

In a methodological sense, the invention provides methods of inhibiting DNA methylation-dependent repression. These methods include the steps of contacting a cell with a test molecule, where the test molecule includes an amino acid sequence at least 100 contiguous amino acids of MBD2-CTH1 or MDB2-CTH2 in length; and determining the ability of the test compound to inhibit DNA methylation-dependent repression, wherein the DNA methylation-dependent repression is mediated by a methyled-CpG binding domain protein or a histone deacetylase protein complex. The invention also provides methods of inhibiting DNA methylation-dependent repression, which includes the steps of: contacting a cell with a test molecule that includes an amino acid sequence of MBD2-CTH1 or MDB2-CTH2; and determining the ability of said the compound to inhibit DNA methylation-dependent repression, where DNA methylation-dependent repression is mediated by a methyled-CpG binding domain protein or a histone deacetylase protein complex.

In addition, the invention contemplates methods for decreasing the amount of MBD2 protein that can bind a sample of methylated DNA, which includes the following step: allowing an effective amount of MBD2-CTH1 peptide to assemble into repressor complexes with a sample of methylated DNA. According to this method, the MBD2-CTH1 interferes with the ability of an MBD2 protein to form repressor complexes.

These and other embodiments are more fully described below.

### BRIEF DESCRIPTION OF THE FIGURES

FIG. 1 depicts the role of MBD2/MBD3 in methylation-dependent transcriptional repression

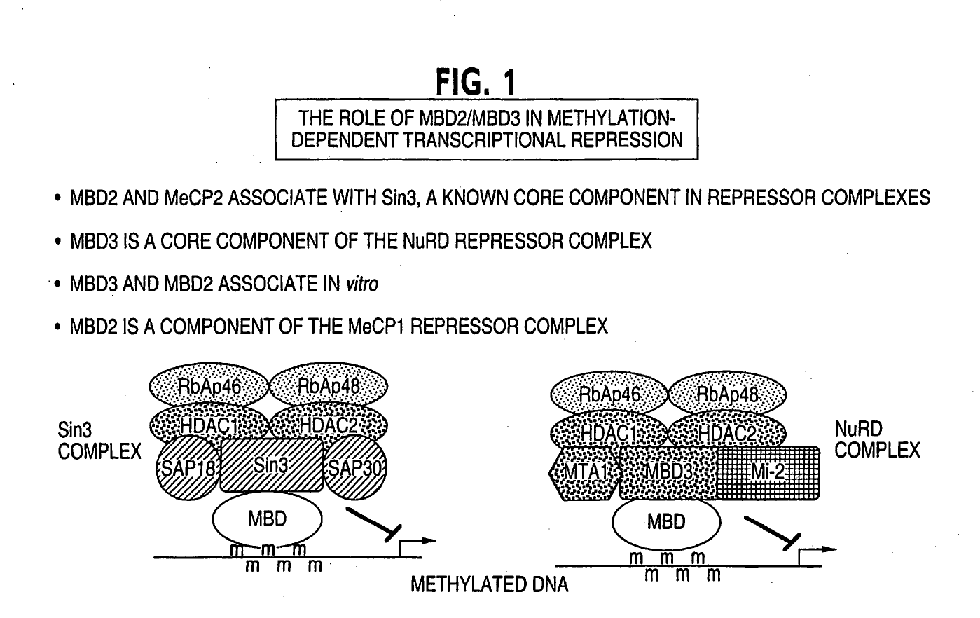

FIG. 2 depicts methylation as a pharmaceutical target

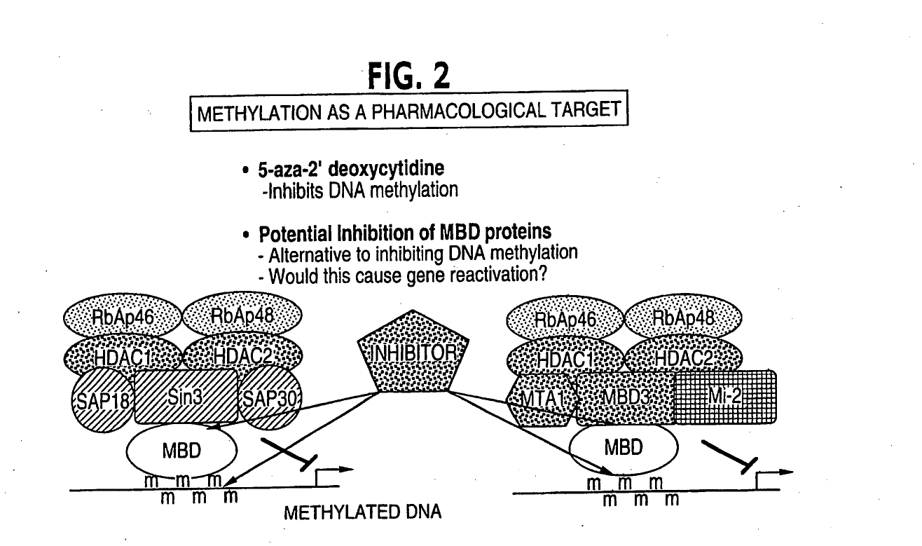

FIG. 3 describes a family of Methyl-CpG binding proteins

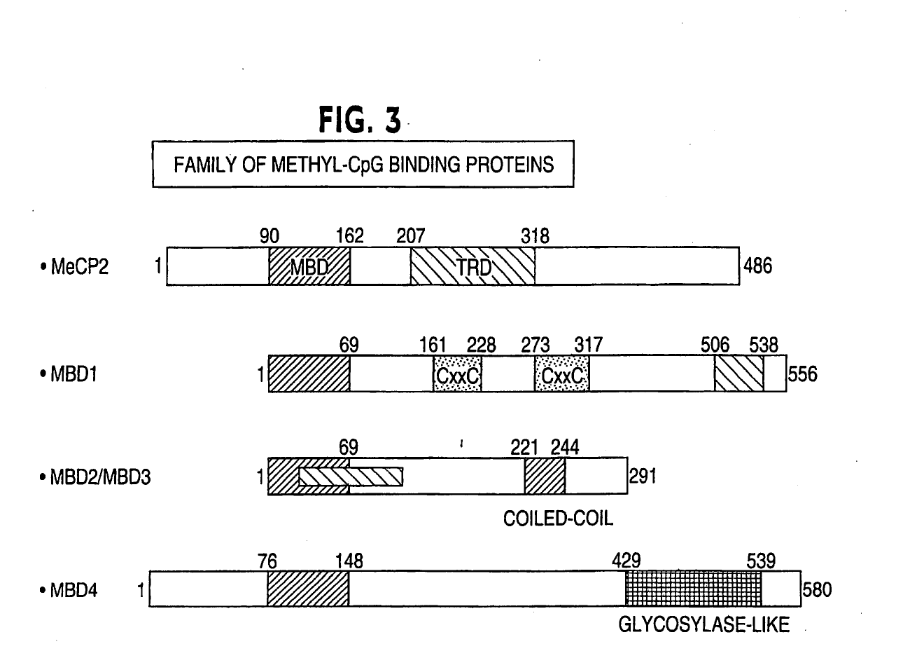

FIG. 4 provides the amino acid sequence of two homologs of MBD2

FIG. 5 describes a homology relationship between MBD2 and two homologs thereof

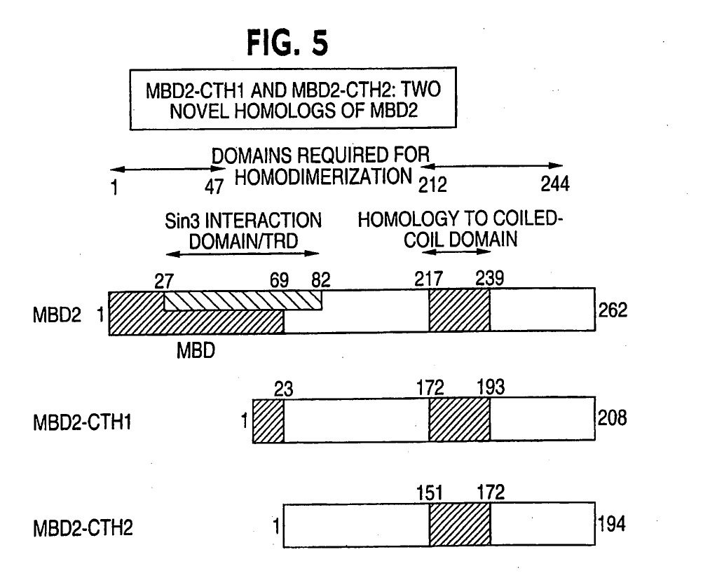

FIG. 6 is a gel illustrating that one MBD2 homolog is expressed in testis

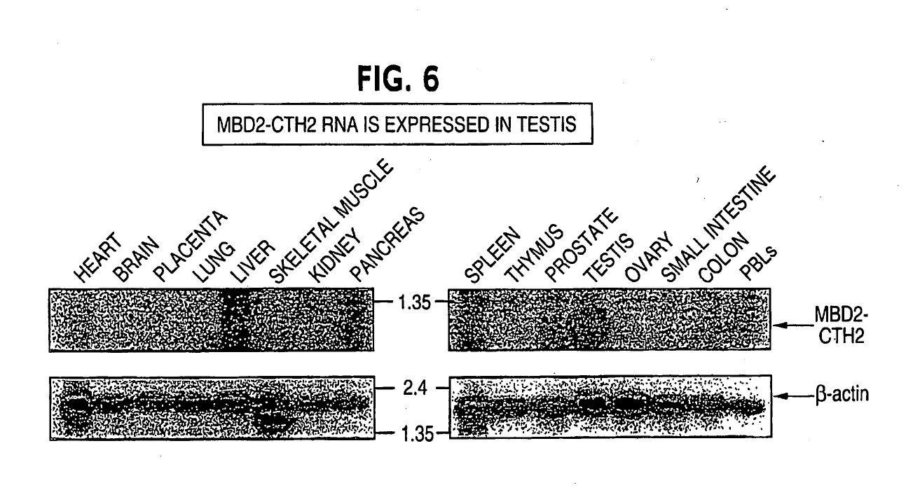

FIG. 7 is a gel illustrating that two MBD2 homologs do not bind methylated DNA

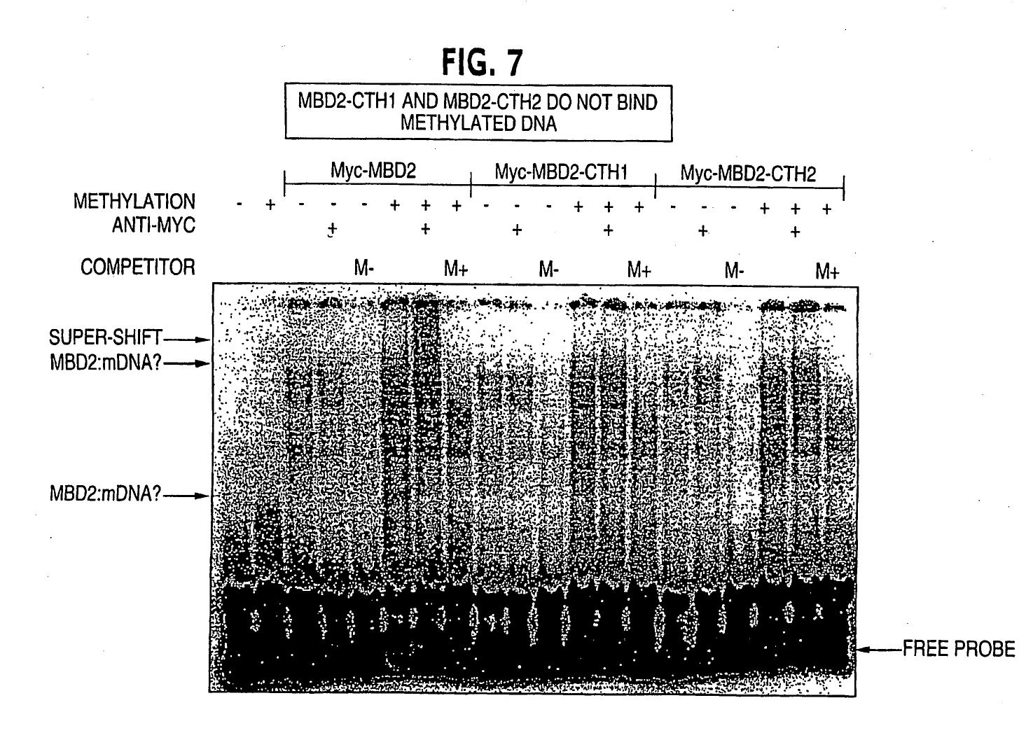

FIG. 8 is a picture illustrating that two MBD2 homologs localize to the nucleus

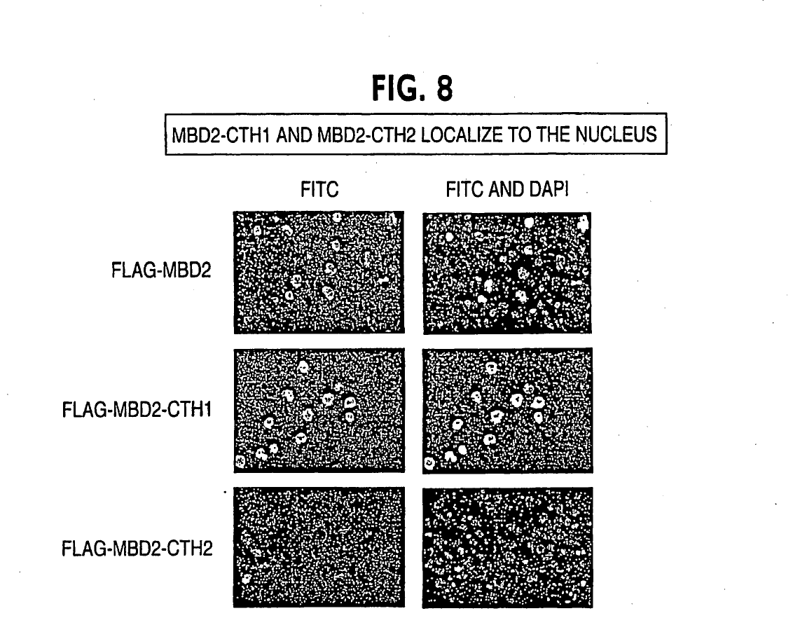

FIG. 9 shows that MBD2 assembles into HDAC2 complexes

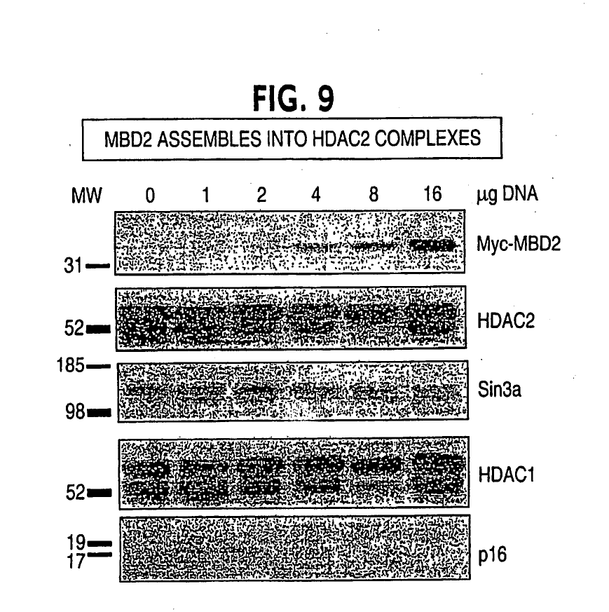

FIG. 10 shows that MBD2-CTH1 assembles into HDAC2 complexes

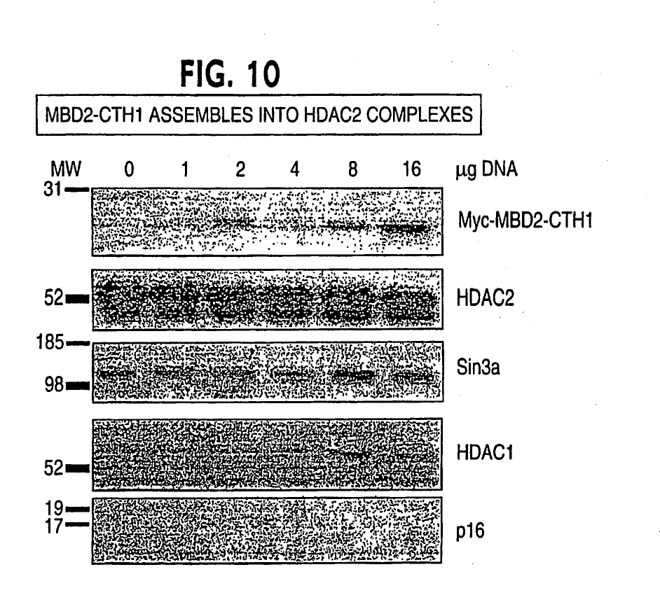

FIG. 11 shows that MBD2-CTH2 assembles into HDAC2 complexes

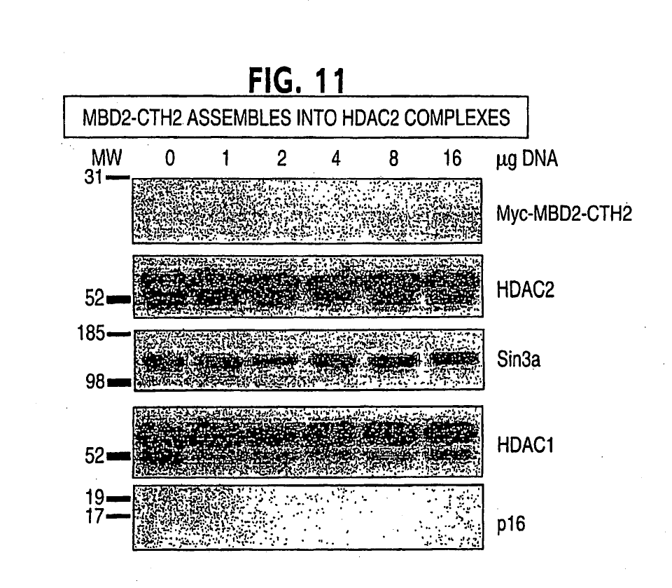

FIG. 12 is a summary of properties of both MBD2-CTH1 and MBD2-CTH2

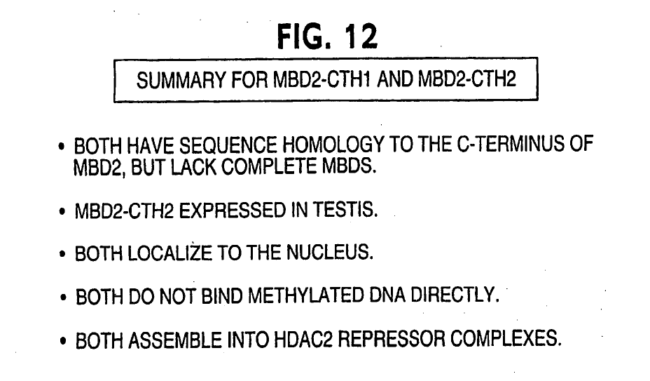

FIG. 13 is a drawing proposing how MBD2-CTH1 and MBD2-CTH2 may be components of repressor complexes.

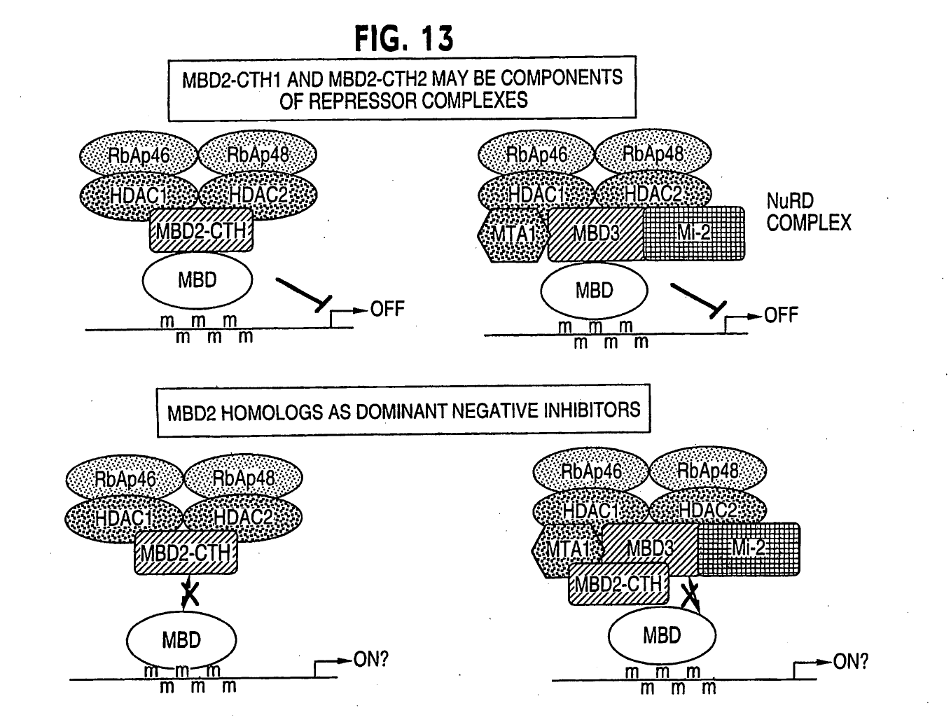

### DETAILED DESCRIPTION

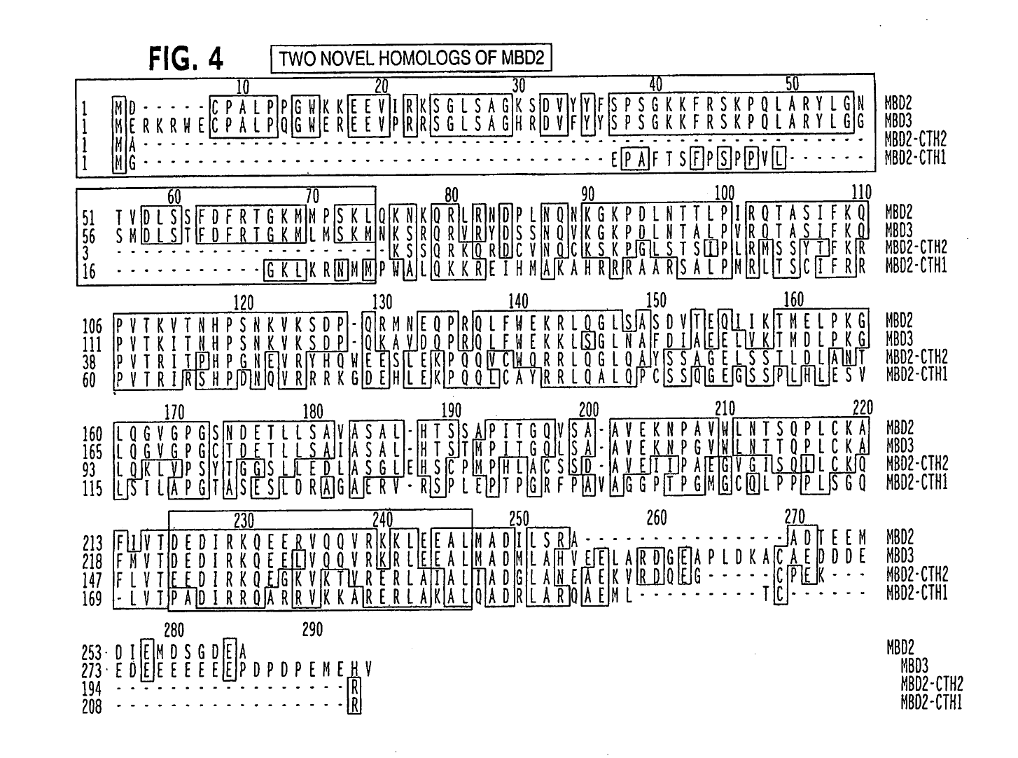

Due to recent elucidation of MDB proteins' role in transcriptional repression, the ability to inhibit MBD proteins represents a potential strategy for reactivating methylation-silenced genes. The present inventors have discovered two new homologs of MBD2. These novel proteins, shown in FIG. 4, lack domains required for binding to methylated DNA, but share extensive sequence identity with the C-terminal region of MBD2. These proteins are denoted as MBD2-C-Terminal Homolog 1 (MBD2-CTH1) and MBD2-C-Terminal Homolog 2 (MBD2-CTH2).

Since the MBD2-CTH1 and MBD2-CTH2 proteins are very similar to MBD2, the skilled worker would expect that the new homologs are able to mimic certain aspects of the activity of MBD2. For example, it is possible that MBD2-CTH1 and MBD2-CTH2 could assemble into transcriptional repressor complexes in the place of MBD2. Since MBD2-CTH1 and MBD2-CTH2 lack MBDs, complexes containing the homologs are unlikely to interact with methylated DNA. MBD2-CTH1 and MBD2-CTH2 apparently act as dominant negative inhibitors of MBD2 and impair transcriptional repression at methylated DNA sites.

DNA and peptide molecules of the invention are represented by the following sequences:

MBD2-CTH1 DNA: (SEQ ID NO: 1)
GGCACGAGGT CAAGAGTGGG GTCAGCAAGA GAAACTCTAC
GGCTATGGGA GAGCCTGCGT TCACCTCTTT TCCGAGCCCA
CCTGTTCTGG GGAAGCTCAA AAGAAACATG ATGCCCTGGG
CTTTACAGAA GAAACGAGAA ATCCACATGG CCAAGGCCCA
TCGGAGACGA GCTGCGAGGT CTGCTCTCCC CATGAGACTC
ACCAGCTGCA TCTTCCGGAG GCCGGTGACA AGGATCAGGT
CTCATCCTGA CAACCAGGTC AGACGCAGAA AAGGGGACGA
GCACCTGGAG AAGCCGCAGC AACTCTGCGC CTACCGGAGA
CTGCAGGCCC TGCAGCCCTG CAGCAGCCAA GGAGAAGGTT
CAAGTCCACT GCATTTGGAG AGCGTCTTAA GTATCCTTGC
ACCGGGGACG GCCAGTGAAT CTCTGGACAG GGCTGGTGCT
GAGCGTGTGC GCAGCCCGCT TGAGCCCACC CCTGGGCGGT
TTCCAGCTGT GGCAGGGGGG CCAACCCCAG GAATGGGTTG
TCAGCTCCCA CCGCCCCTCT CTGGCCAATT GGTGACTCCT
GCAGATATCC GGAGACAGGC CAGGAGGGTG AAGAAAGCCA
GGGAGAGACT GGCCAAGGCC TTGCAGGCAG ACAGGCTGGC
CAGGCAGGCA GAAATGCTGA CATGTAGATG AAGCGCAGTC
CTGGGCTTTC GGTCCCTTTC TTTTAATGCC CATCCTCATT
CCTACTCTGA ATTGTCACAC TTTTCCCTTC CCCACCAGTT
CTTTAATAAA AGTATTTGAA AGGCAAAAAA AAAAAAAAAA
AAAAAAAGAT TT

The DNA sequence for MBD2-CTH1 is 812 nucleotides in length.

MBD2-CTH1 Protein: (SEQ ID NO: 2)
MGEPAFTSFP SPPVLGKLKR NMMPWALQKK REIHMAKAHR
RRAARSALPM RLTSCIFRRP VTRIRSHPDN QVRRRKGDEH
LEKPQQLCAY RRLQALQPCS SQGEGSSPLH LESVLSILAP
GTASESLDRA GAERVRSPLE PTPGRFPAVA GGPTPGMGCQ
LPPPLSGQLV TPADIRRQAR RVKKARERLA KALQADRLAR
QAEMLTCR

The polypeptide sequence for MBD2-CTH1 is 208 amino acids in length.

MBD2-CTH2 DNA: (SEQ ID NO: 3)
AGCAGTGATC ATATGACGGT GGTAGCCATG GCGTCACAGA
GAACA GGGAC CTTCCAAGTT TGAAGTTTTA CAAGGCAGGT
GTGATAAGTG TGTAAGAGGA AAAGAAGTGT GATGGCCAAG
AGTTCACAGA GGAAGCAACG TGACTGTGTA AACCAATGCA
AATCAAAGCC TGGCTTGAGC ACCTCAATCC CTTTGAGAAT
GTCCAGTTAC ACATTCAAGA GGCCAGTAAC GAGAATTACA
CCCCATCCTG GCAATGAGGT CAGATACCAT CAATGGGA GG
AGAGCTTGGA GAAGCCTCAG CAGGTCTGCT GGCAGAGGAG
ACTGCAGGGA CTCCAGGCTT ACAGCAGTGC AGGAGAACTT
TCAAGCACTT TGGATCTTGC CAATACCTTG CAAAAACTTG
TCCCTAGTTA CACAGGTGGA TCTCTGCTGG AGGATCTTGC
CAGTGGTCTG GAGCACTCCT GCCCCATGCC CCACCTTGCC
TGCTCTTCAG ATGCGGTGGA GATAATTCCT GCAGAGGGAG
TGGGTATCTC GCAGCTCCTC TGCAAACAAT TTCTGGTCAC
TGAGGAAGAT ATCAGGAAAC AGGAAGGGAA AGTGAAGACA
GTCAGAGAGA GACTCGCAAT AGCACTGATT GCGGATGGAC
TCGCTAATGA GGCAGAGAAA GTGAGAGACC AAGAAGGCTG
TCCTGAAAAA CGCTAAGAAA AAAAGGGAAG ATAGTGCAGA
TGAAATAAAG TGTAATCCTT TAT TAACATC TCAAAAAAAA
AAAAAAAAAA AAAAAAAAAA AAAAAAAAAA

The DNA sequence for MBD2-CTH2 is 790 nucleotidesin length.

MBD2-CTH2 Protein: (SEQ ID NO: 4)
MAKSSQRKQR DCVNQCKSKP GLSTSIPLRM SSYTFKRPVT
RITPHPGNEV RYHQWEESLE KPQQVCWQRR LQGLQAYSSA
GELSSTLDLA NTLQKLV PSY TGGSLLEDLA SGLEHSCPMP
HLACSSDAVE IIPAEGVGIS QLLCKQFLVT EEDIRKQEGK
VKTVRERLAI ALIADGLANE AEKVRDQEGC PEKR

The polypeptide sequence for MBD2-CTH1 is 194 amino acids in length.

Characterization of MBD2-CTH1 and MBD2-CTH2

The present inventors characterized the MBD2-CTH1 and MBD2-CTH2 proteins to determine whether they can inhibit or compete with MBD2, which is involved in binding methylated DNA. The use of MBD2-CTH1 and/or MBD2-CTH2 would, accordingly, provide new methods for relieving methylation-dependent gene repression and, therefore, permit the development of drugs to prevent the repression of tumor suppressor genes.

Methylated DNA binding proteins, typified by MBD2, are thought to repress transcription (Nan et al. 1997; Ng et al. 2000; Ng et al. 1999) and are defined structurally by an N-terminal domain that specifically interacts with methylated DNA (Cross et al. 1997; Hendrich and Bird 1998; Lewis et al. 1992). The novel proteins of the invention, MBD2-CTH1 and MBD2-CTH2, share amino acid sequence homology to the C-terminal region of MBD2 and MBD3 (Hendrich and Bird 1998), suggesting a role or function similar to MBD2. In fact, the protein sequences of MBD2-CTH1 and MBD2-CTH2 share significant similarities with MBD2 and MBD3 and contain a domain important for interacting with other proteins, but do not have complete MBDs.

Although MBD2-CTH1 and MBD2-CTH2 do not have MBDs, they have sufficient sequence similarity to MBD2 that they are expected to share some functions with MBD2. For example, the homologs would be expected to localize to the nucleus within a cell. Investigating cellular localization of the novel homologs by immunofluorescence staining indicated that MBD2-CTH1 and MBD2-CTH2 accumulated in the nucleus in a manner similar to MBD2. Since MBD2-CTH1 and MBD2-CTH2 do not have complete MBDs, their accumulation in the nucleus suggests that the MBD domain is not required for nuclear localization. The nuclear localization of MBD2-CTH1 and MBD2-CTH2 also suggested that they assemble into chromatin-repressing complexes.

To determine what regions of the MBD2, MBD2-CTH1 and MBD2-CTH2 proteins are important for nuclear localization, proteins were created having portions that were deleted. These mutant proteins were examined in the nucleus using immunofluorescence staining. Although these mutant proteins lacked the C-terminal portions, they exhibited the same nuclear staining as full-length MBD2, MBD2-CTH1 and MBD2-CTH2 proteins. When only the MBD of the MBD2 protein was analyzed, however, diffuse cellular staining was observed. These data suggest that a portion of MBD2 after (i.e. C-terminal to) the MBD is important for nuclear localization. At least a portion of the C-terminal region is not needed for nuclear localization, however.

One function of MBD2 is to bind methylated DNA via its MBD (Hendrich and Bird 1998). As indicated above, although MBD2-CTH1 and MBD2-CTH2 are similar to MBD2, they do not contain complete MBDs, so it would not be expected that the homologs bind methylated DNA. Electrophoretic mobility shift assays (EMSAs) confirmed this assumption, indicating that MBD2-CTH1 and MBD2-CTH2 were indeed incapable of binding methylated DNA.

Another function of MBD2 is to assemble, e.g. by self-assembly by recruiting other molecules into transcriptional repressor complexes. MBD2 is present in the MeCP1 repressor complex and interacts with the Sin3 and NuRD repressor complexes (Boeke et al. 2000; Ng et al. 1999; Zhang et al. 1999). This evidence indicates that methyl-CpG binding proteins are an integral part of transcriptional repression.

Despite their lack of MBDs, MBD2-CTH1 and MBD2-CTH2 were examined to see of they could assemble into repressor complexes, such as by isolating the histone deacetlyase HDAC2 and maintaining the integrity of any complex that contained HDAC2. The complexes were analyzed for the presence of MBD2, MBD2-CTH1 and MBD2-CTH2. As expected, HDAC2 complexes that were obtained from cells expressing MBD2 contained MBD2, as well as Sin3. In parallel experiments, association of MBD2-CTH1 and MBD2-CTH2 with HDAC2 and Sin3 was observed.

These observations indicate that MBD2-CTH1 and MBD2-CTH2 can assemble into repressor complexes, despite their inability to bind methylated DNA. Since these homologs assemble into repressor complexes, they likely function as dominant-negative competitors of MBD2 and provide a new method for inhibiting methylation-dependent repression because, as described more fully herein, MBD2-CTH1 and MBD2-CTH2 do not have MBDs. Lacking MBDs, the MBD2-CTH1 and MBD2-CTH2 proteins are not expected to recruit transcriptional repressor complexes to methylated DNA. Instead, these MBD2 homologs may compete with MBD2 in the complexes and, thus, prevent potential repressor complexes from binding methylated DNA.

Role of MBD2-CTH1 and MBD2-CTH2 in Transcriptional Repression Complexes.

At least one of the novel proteins is able to compete with MBD2 in repressor complexes. To examine whether MBD2-CTH1 and MBD2-CTH2 compete with MBD2 in repressor complexes, HDAC2 complexes were isolated in the context of a constant amount of MBD2 protein and increasing amounts of MBD2-CTH1 or MBD2-CTH2 proteins. As the amount of MBD2-CTH1 and MBD2-CTH2 protein increased, the amount of MBD2-CTH1 or MBD2-CTH2 present in the HDAC2 complex increased. The amount of MBD2 present in the HDAC2 complexes decreased as the MBD2-CTH1 protein increased, but did not follow the same pattern in the context of increasing MBD2-CTH2. These data indicate that there is competition between MBD2 and MBD2-CTH1, but not MBD2 and MBD2-CTH2. In other words, the data indicate that MBD2-CTH1, but not MBD2-CTH2, competes with MBD2 in repressor complexes. This difference in activities for MBD2-CTH1 and MBD2-CTH2 is surprising, because the homologs have such high sequence identity to each other. Yet, these data suggest that these two homologs may interact with different proteins in the repressor complexes.

The presence of MBD2-CTH1 and MBD2-CTH2 in repressor complexes indicates that the homologs influence transcription. If the homologs act as inhibitors of MBD2, then they may reverse, inhibit or otherwise interfere with the repression of methylation-silenced genes.

By way of example, the ability of MBD2-CTH1 and MBD2-CTH2 to change transcription of genes can be analyzed by the expression pattern of the p16 gene by northern blot analysis and luciferase assay. It is known that the p16 gene is repressed by methylation in many colon cancer cell lines. If the homologs inhibit MBD2 mediated repression, then expression of MBD2-CTH1 or MBD2-CTH2 protein in cells with methylation-silenced p16 should result in expression of p16. These results can be compared with results from cells treated with the demethylating agent aza-dC, allowing evaluation of whether the mechanisms for inhibition are similar. In addition, the global transcriptional changes in cells with MBD2-CTH1 or MBD2-CTH2 can be examined by microarray analysis. Microarray analysis allows examination of the expression changes of approximately 13,000 genes, some of which are known to be methylation silenced in colon cancer. These data can be compared to microarray data obtained from cells treated with aza-dC.

To determine what regions of the MBD2, MBD2-CTH1 and MBD2-CTH2 proteins are important for assembling into the repressor complexes, proteins with portions that are deleted can be created. These mutant proteins can be tested to determine, for example, if they are capable of assembling into repressor complexes. It is possible that MBD2, MBD2-CTH1 and MBD2-CTH2 interact with each other, but the homologs may lack a domain important for protein interactions mediated by MBD2 that would result in the loss of normal MBD2 function. Alternatively, the homologs may interact with other proteins in the repressor complexes in the same manner as MBD2 and, as a result, directly compete with MBD2 for binding partners. It is possible that the regions important for assembling into repressor complexes also dictate whether the protein is located in the nucleus, since these proteins are relatively small (could otherwise freely diffuse in and out of the nucleus), yet they appear to remain nuclear.

### METHODS OF THE INVENTION

DNA and peptide molecules of the invention can be applied to inhibit DNA methylation-dependent repression. In one aspect, the invention provides a method that includes the steps of contacting a cell with a test molecule that contains an amino acid sequence of at least 100 contiguous amino acids of MBD2-CTH1 or MDB2-CTH2 peptide; and determining the ability of the test compound to inhibit DNA methylation-dependent repression. In this regard, any DNA methylation-dependent repression is mediated by a methyled-CpG binding domain protein and/or histone deacetylase protein complexes.

In another aspect, the invention provides methods of decreasing the amount of MBD2 protein that can bind a sample of methylated DNA. This method contemplates at least the following steps: placing an effective amount of MBD2-CTH1 peptide in close proximity to the methylated DNA sample; and allowing the MBD2-CTH1 peptide to assemble into repressor complexes with the methylated DNA. By forming such repressor complexes, the MBD2-CTH1 consequently interferes with the ability of an MBD2 protein to form such repressor complexes. By “effective amount” of MBD2-CTH1 peptide is meant an amount that is sufficient to bring about a desirable decrease in the amount of MBD2 protein that can bind a given sample of methylated DNA. The skilled worker will, through routine experimentation, be able to determine (i) the amount of MBD2-CTH1 peptide needed, as well as (ii) the desired decrease in the amount of MBD2 protein that can bind a given sample of methylated DNA.

### REFERENCES

American Cancer Society. (2001). “Cancer facts & figures 2001.” Amercian Cancer Society, Inc. Atlanta, Ga.
Boeke, J., Ammerpohl, O., Kegel, S., Moehren, U., and Renkawitz, R. (2000). “The minimal repression domain of MBD2b overlaps with the methyl-CpG-binding domain and binds directly to sin3A.” J Biol Chem, 275(45), 34963-7.
Cross, S. H., Meehan, R. R., Nan, X., and Bird, A. (1997). “A component of the transcriptional repressor MeCP1 shares a motif with DNA methyltransferase and HRX proteins.” Nat Genet, 16(3), 256-9.
Cunningham, J. M., Christensen, E. R., Tester, D. J., Kim, C. Y., Roche, P. C., Burgart, L. J., and Thibodeau, S. N. (1998). “Hypermethylation of the hMLH1 promoter in colon cancer with microsatellite instability.” Cancer Res, 58(15), 3455-60.
Hendrich, B., and Bird, A. (1998). “Identification and characterization of a family of mammalian methyl-CpG binding proteins.” Mol Cell Biol, 18(11), 6538-47.
Herman, J. G., Latif, F., Weng, Y., Lerman, M. I., Zbar, B., Liu, S., Samid, D., Duan, D. S., Gnarra, J. R., Linehan, W. M., and et al. (1994). “Silencing of the VHL tumor-suppressor gene by DNA methylation in renal carcinoma.” Proc Natl Acad Sci U S A, 91(21), 9700-4.
Herman, J. G., Merlo, A., Mao, L., Lapidus, R. G., Issa, J. P., Davidson, N. E., Sidransky, D., and Baylin, S. B. (1995). “Inactivation of the CDKN2/p16/MTS1 gene is frequently associated with aberrant DNA methylation in all common human cancers.” Cancer Res, 55(20), 4525-30.
Herman, J. G., Umar, A., Polyak, K., Graff, J. R., Ahuja, N., Issa, J. P., Markowitz, S., Willson, J. K., Hamilton, S. R., Kinzler, K. W., Kane, M. F., Kolodner, R. D., Vogelstein, B., Kunkel, T. A., and Baylin, S. B. (1998). “Incidence and functional consequences of hMLH1 promoter hypermethylation in colorectal carcinoma.” Proc Natl Acad Sci USA, 95(12), 6870-5.
Hiltunen, M. O., Alhonen, L., Koistinaho, J., Myohanen, S., Paakkonen, M., Marin, S., Kosma, V. M., and Janne, J. (1997). “Hypermethylation of the APC (adenomatous polyposis coli) gene promoter region in human colorectal carcinoma.” Int J Cancer, 70(6), 644-8.
Jones, P. L., Veenstra, G. J., Wade, P. A., Vermaak, D., Kass, S. U., Landsberger, N., Strouboulis, J., and Wolffe, A. P. (1998). “Methylated DNA and MeCP2 recruit histone deacetylase to repress transcription.” Nat Genet, 19(2), 187-91.
Kane, M. F., Loda, M., Gaida, G. M., Lipman, J., Mishra, R., Goldman, H., Jessup, J. M., and Kolodner, R. (1997). “Methylation of the hMLH1 promoter correlates with lack of expression of hMLH1 in sporadic colon tumors and mismatch repair-defective human tumor cell lines.” Cancer Res, 57(5), 808-11.
Lewis, J. D., Meehan, R. R., Henzel, W. J., Maurer-Fogy, I., Jeppesen, P., Klein, F., and Bird, A. (1992). “Purification, sequence, and cellular localization of a novel chromosomal protein that binds to methylated DNA.” Cell, 69(6), 905-14.
Merlo, A., Herman, J. G., Mao, L., Lee, D. J., Gabrielson, E., Burger, P. C., Baylin, S. B., and Sidransky, D. (1995). “5′ CpG island methylation is associated with transcriptional silencing of the tumour suppressor p16/CDKN2/MTS1 in human cancers.” Nat Med, 1(7), 686-92.
Nan, X., Campoy, F. J., and Bird, A. (1997). “MeCP2 is a transcriptional repressor with abundant binding sites in genomic chromatin.” Cell, 88(4), 471-81.
Ng, H. H., Jeppesen, P., and Bird, A. (2000). “Active repression of methylated genes by the chromosomal protein MBD1.” Mol Cell Biol, 20(4), 1394-406.
Ng, H. H., Zhang, Y., Hendrich, B., Johnson, C. A., Turner, B. M., Erdjument-Bromage, H., Tempst, P., Reinberg, D., and Bird, A. (1999). “MBD2 is a transcriptional repressor belonging to the MeCP1 histone deacetylase complex.” Nat Genet, 23(1), 58-61.
Tatematsu, K. I., Yamazaki, T., and Ishikawa, F. (2000). “MBD2-MBD3 complex binds to hemi-methylated DNA and forms a complex containing DNMT1 at the replication foci in late S phase.” Genes Cells, 5(8), 677-88.
Veigl, M. L., Kasturi, L., Olechnowicz, J., Ma, A. H., Lutterbaugh, J. D., Periyasamy, S., Li, G. M., Drummond, J., Modrich, P. L., Sedwick, W. D., and Markowitz, S. D. (1998). “Biallelic inactivation of hMLH1 by epigenetic gene silencing, a novel mechanism causing human MSI cancers.” Proc Natl Acad Sci USA, 95(15), 8698-702.
Wade, P. A., Gegonne, A., Jones, P. L., Ballestar, E., Aubry, F., and Wolffe, A. P. (1999). “Mi-2 complex couples DNA methylation to chromatin remodelling and histone deacetylation.” Nat Genet, 23(1), 62-6.
Zhang, Y., Ng, H. H., Erdjument-Bromage, H., Tempst, P., Bird, A., and Reinberg, D. (1999). “Analysis of the NuRD subunits reveals a histone deacetylase core complex and a connection with DNA methylation.” Genes Dev, 13(15), 1924-35.

1. A purified nucleic acid molecule comprising at least 200 contiguous nucleotides of a nucleic acid sequence encoding MBD2-CTH1 (SEQ ID NO: 2) or MDB2-CTH2 (SEQ ID NO: 4), or a complementary strand of said nucleic acid sequence.

2. A purified nucleic acid molecule encoding MBD2-CTH1 (SEQ ID NO: 2) or MDB2-CTH2 (SEQ ID NO: 4), or a complementary strand of said nucleic acid sequence.

3. A purified polypeptide comprising at least 67 contiguous amino acids of the amino acid sequence of MBD2-CTH1 (SEQ ID NO: 2) or MDB2-CTH2 (SEQ ID NO: 4).

4. A purified nucleic acid molecule comprising a nucleic acid sequence that encodes the amino acid sequence of MBD2-CTH1 (SEQ ID NO: 2)or MDB2-CTH2 (SEQ ID NO: 4).

5. A method of inhibiting DNA methylation-dependent repression comprising the steps of:
a) contacting a cell with a test molecule, wherein said test molecule comprises an amino acid sequence comprising at least 67 contiguous amino acids of MBD2-CTH1 or MDB2-CTH2, and
b) determining the ability of said test compound to inhibit DNA methylation-dependent repression,
wherein said DNA methylation-dependent repression is mediated by one or more proteins selected form the group consisting of methyled-CpG binding domain proteins and histone deacetylase protein complexes.

6. A method of inhibiting DNA methylation-dependent repression comprising the steps of:
a) contacting a cell with a test molecule, wherein said test molecule comprises an amino acid sequence of MBD2-CTH1 or MDB2-CTH2 and
b) determining the ability of said test compound to inhibit DNA methylation-dependent repression,
wherein said DNA methylation-dependent repression is mediated by one or more proteins selected form the group consisting of methyled-CpG binding domain proteins and histone deacetylase protein complexes.

7. A purified DNA molecule comprising a DNA sequence selected from the group consisting of SEQ ID NO: 1, a complementary strand of SEQ ID NO: 1, SEQ ID NO: 3, and a complementary strand of SEQ ID NO: 3.

8. A method of decreasing the amount of MBD2 protein that can bind a sample of methylated DNA, comprising allowing an effective amount of MBD2-CTH1 peptide to assemble into repressor complexes with a sample of methylated DNA, wherein said MBD2-CTH1 interferes with the ability of an MBD2 protein to form said repressor complexes.


SEQUENCE LISTING (6 sequences)

SEQ ID NO: 1 - DNA, 812 bp, Homo sapiens
ggcacgaggtcaagagtggggtcagcaagagaaactctacggctatgggagagcctgcgttcacctcttttccgagcccacctgttctggggaagctcaaaagaaacatgatgccctgggctttacagaagaaacgagaaatccacatggccaaggcccatcggagacgagctgcgaggtctgctctccccatgagactcaccagctgcatcttccggaggccggtgacaaggatcaggtctcatcctgacaaccaggtcagacgcagaaaaggggacgagcacctggagaagccgcagcaactctgcgcctaccggagactgcaggccctgcagccctgcagcagccaaggagaaggttcaagtccactgcatttggagagcgtcttaagtatccttgcaccggggacggccagtgaatctctggacagggctggtgctgagcgtgtgcgcagcccgcttgagcccacccctgggcggtttccagctgtggcaggggggccaaccccaggaatgggttgtcagctcccaccgcccctctctggccaattggtgactcctgcagatatccggagacaggccaggagggtgaagaaagccagggagagactggccaaggccttgcaggcagacaggctggccaggcaggcagaaatgctgacatgtagatgaagcgcagtcctgggctttcggtccctttcttttaatgcccatcctcattcctactctgaattgtcacacttttcccttccccaccagttctttaataaaagtatttgaaaggcaaaaaaaaaaaaaaaaaaaaaaagattt

SEQ ID NO: 2 - protein, 208 aa, Homo sapiens [sequence omitted]

SEQ ID NO: 3 - DNA, 790 bp, Homo sapiens
agcagtgatcatatgacggtggtagccatggcgtcacagagaacagggaccttccaagtttgaagttttacaaggcaggtgtgataagtgtgtaagaggaaaagaagtgtgatggccaagagttcacagaggaagcaacgtgactgtgtaaaccaatgcaaatcaaagcctggcttgagcacctcaatccctttgagaatgtccagttacacattcaagaggccagtaacgagaattacaccccatcctggcaatgaggtcagataccatcaatgggaggagagcttggagaagcctcagcaggtctgctggcagaggagactgcagggactccaggcttacagcagtgcaggagaactttcaagcactttggatcttgccaataccttgcaaaaacttgtccctagttacacaggtggatctctgctggaggatcttgccagtggtctggagcactcctgccccatgccccaccttgcctgctcttcagatgcggtggagataattcctgcagagggagtgggtatctcgcagctcctctgcaaacaatttctggtcactgaggaagatatcaggaaacaggaagggaaagtgaagacagtcagagagagactcgcaatagcactgattgcggatggactcgctaatgaggcagagaaagtgagagaccaagaaggctgtcctgaaaaacgctaagaaaaaaagggaagatagtgcagatgaaataaagtgtaatcctttattaacatctcaaaaaaaaaaaaaaaaaaaaaaaaaaaaaaaaaaaaaa

SEQ ID NO: 4 - protein, 194 aa, Homo sapiens
MAKSSQRKQRDCVNQCKSKPGLSTSIPLRMSSYTFKRPVTRITPHPGNEVRYHQWEESLEKPQQVCWQRRLQGLQAYSSAGELSSTLDLANTLQKLVPSYTGGSLLEDLASGLEHSCPMPHLACSSDAVEIIPAEGVGISQLLCKQFLVTEEDIRKQEGKVKTVRERLAIALIADGLANEAEKVRDQEGCPEKR

SEQ ID NO: 5 - protein, 262 aa, Homo sapiens
MDCPALPPGWKKEEVIRKSGLSAGKSDVYYFSPSGKKFRSKPQLARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDEA

SEQ ID NO: 6 - protein, 291 aa, Homo sapiens
MERKRWECPALPQGWEREEVPRRSGLSAGHRDVFYYSPSGKKFRSKPQLARYLGGSMDLSTFDFRTGKMLMSKMNKSRQRVRYDSSNQVKGKPDLNTALPVRQTASIFKQPVTKITNHPSNKVKSDPQKAVDQPRQLFWEKKLSGLNAFDIAEELVKTMDLPKGLQGVGPGCTDETLLSAIASALHTSTMPITGQLSAAVEKNPGVWLNTTQPLCKAFMVTDEDIRKQEELVQQVRKRLEEALMADMLAHVEELARDGEAPLDKACAEDDDEEDEEEEEEEPDPDPEMEHV